(19) United States
(12) Patent Application Publication
(10) Pub. No.: US 2020/0353246 A1
(43) Pub. Date: Nov. 12, 2020

(54) SKIN ATTACHMENT CONNECTING DEVICE FOR APPLYING MICRO-ELECTRIC CURRENT FOR CELL MOVEMENT OF SKIN

(71) Applicant: CELLOGIN INC., Wonju-si (KR)

(72) Inventors: Han Sung KIM, Seoul (KR); Seung Kwan CHO, Goyang-si (KR); Chang Yeon WON, Sinbanpo-ro (KR); Chi Hwan LEE, Seongnam-si (KR); Jong Kyu PARK, Yongin-si (KR)

(21) Appl. No.: 16/640,313

(22) PCT Filed: Aug. 2, 2018
(86) PCT No.: PCT/KR2018/008802

§ 371 Date: Feb. 19, 2020

(30) Foreign Application Priority Data
Aug. 21, 2017 (KR) 10-2017-0105750

(51) Int. Cl. A61N 1/32; A61N 1/04; A61N 1/30

(52) U.S. Cl. CPC A61N 1/328; A61N 1/303; A61N 1/325; A61N 1/0428

(57) ABSTRACT

The present invention relates to a skin attachment connecting device and, more specifically, to a skin attachment connecting device for applying a micro-electric current for a cell movement of the skin, wherein a protrusion part comes into contact with a skin attachment while the connecting device formed in a tong shape picks up the attachment formable in various shapes, such that a micro-electric current is applied to the skin attachment. In order to achieve the purpose as described above, a connecting device for applying power transferred from a power supply device to a skin attachment, according to the present invention, is formed in a tong shape and comprises, therein, a connection part for applying power transferred from the power supply device to the skin attachment while contacting the skin attachment.

### TECHNICAL FIELD

The present invention relates to a connecting device for a skin attaching article and, specifically, to a connecting device for a skin attaching article in which the connecting device, which is shaped as a clamp, clamps the skin attaching article, which may be formed in various shapes and, in the state, a protrusion contacts the skin attaching article and applies a micro current for skin cell exercise.

### BACKGROUND ART

Generally, if a current with a strength close to the bio-electricity is applied to the skin, the cell (skin) exercise and iontophoresis effects both may occur.

Iontophoresis is a technique for delivery active ingredients good for skin to the skin by allowing a micro current to flow to the skin. As a skin attaching article with active skin ingredients applied thereto flow to the skin, a circuit is created between the skin and the skin attaching article, and the active skin ingredients are delivered to the skin via the circuit by the electrical repulsive force, contributing to reducing wrinkles, whitening, or tone-up.

A common massager adopting iontophoresis had a coupling means for connecting to a skin attaching article and a connecting means for receiving electric current from a power supply. When the coupling means and the connecting means are connected together, the massager supplies electric current to the skin attaching article.

Such a conventional skin attaching article connecting means is disclosed in Korean Utility Model Registration No. 20-0403033 (hereinafter, referred to as “conventional art”).

The conventional art includes a wet matrix face mask, a work electrode coupling means, an opposite electrode body contact, and a galvanic ion current generator. The work electrode coupling means is electrically connected to the wet matrix face mask which is attached to the body skin, and the opposite electrode body contact contacts a body portion, so that they are electrically connected together. A galvanic ion current generated from the galvanic ion current generator is applied to the face mask.

In the conventional art, the work electrode coupling means is clamp-shaped and, as coupled to a connection terminal of the face mask, the coupling means is electrically connected. If the coupling means is not properly coupled to the connection terminal, electric current may be difficult to apply. Thus, connecting the coupling means in the precise position is material.

Meanwhile, in such a massager, the face mask with a skin care liquid applied is placed on the face, and then, the coupling means is connected, and electric current is applied to the connected face mask.

However, upon use of this massager, the user may have the skin care liquid which is slippery on his hands and so his fingers may easily slip off the coupling means, rendering it difficult to handle the coupling means.

Thus, to use the coupling means properly, the user needs to wipe the skin care liquid off his hands before handling the coupling means. This is inconvenient.

### DETAILED DESCRIPTION OF THE INVENTION

### Technical Problems

The present invention has been made to address the foregoing problems of the conventional art. According to the present invention, there is provided a connecting device for a skin attaching article in which the connecting device, which is shaped as a clamp, clamps the skin attaching article, which may be formed in various shapes and, in the state, a protrusion contacts the skin attaching article and applies a micro current for skin cell exercise.

### Means to Address the Problems

To achieve the foregoing objectives, according to the present invention, there is provided a connecting device for a skin attaching article, for applying power received from a power supply device to a skin attaching article. The connecting device is shaped as a clamp. The connecting device has a connecting part in an inside thereof. The contacting part contacts the skin attaching article to apply the power received from the power supply device to the skin attaching article.

Meanwhile, a handle of the connecting device has a plurality of anti-slip protrusions projecting from an outside surface.

In this case, each of the anti-slip protrusions has a flat end.

Meanwhile, a coupling hole is formed in the skin attaching article to allow the connecting device to pass therethrough.

Meanwhile, a handle of the connecting device includes an opening formed in the handle towards an upper body and a cover configured to couple to, and decouple from, the opening to close or open an internal space of the handle.

Or, a handle of the connecting device includes an opening formed in an inside of the handle towards an upper body. The opening is configured to be opened or closed by an elastic part used for clamping by the connecting device.

### Effects of the Invention

According to the present invention, the connecting device is shaped as a clamp which enables connection or disconnection to/from the skin attaching article simply by opening or closing the clamp. This allows for easy use. Unlike the conventional art, there is no need for a coupling means for connection to the skin attaching article. Thus, manufacturing costs may be reduced.

According to the present invention, although the connecting device is handled with the user's hands with the slippery skin care liquid on, the anti-slip protrusions may prevent the user's fingers from slipping off, thus enhancing the user's convenience.

### BRIEF DESCRIPTION OF THE DRAWINGS

FIG. 1 is a view illustrating an example of wearing a connecting device according to an embodiment of the present invention;

FIG. 2 is a view illustrating a skin attaching article according to another embodiment of the present invention;

FIG. 3 is a perspective view illustrating a connecting device according to the present invention;

FIG. 4 is a partial perspective view illustrating a connecting device according to the present invention;

FIG. 5 is a side view illustrating a connecting device according to the present invention;

FIG. 6 is a view illustrating anti-slip protrusions according to an embodiment of the present invention;

FIG. 7 is a view illustrating anti-slip protrusions according to another embodiment of the present invention;

FIG. 8 is a partially enlarged photo of prototype anti-slip protrusions according to the present invention;

FIGS. 9, 10, 11, and 12 are views illustrating example handles according to various embodiments of the present invention; and

FIG. 13 is a view illustrating an example of wearing a connecting device according to another embodiment of the present invention.

### DESCRIPTION OF REFERENCE CHARACTERS

| | |
|---|---|
| W: electric wire | S: elastic part |
| 10: connecting device | |
| 11: upper body | 12: lower body |
| 13: connecting part | 14: protrusion |
| 15: coupling part | 16: handle |
| 20: skin attaching article | 21: coupling hole |
| 30: power supply device | |
| 40: anti-slip protrusion | |
| 51, 51a, 51b, 51c: opening part | |
| 52: seating part | |
| 53, 51a, 53b, 53c: cover | |

### BEST MODE TO PRACTICE THE INVENTION

Hereinafter, preferred embodiments of the present invention are described in detail with reference to the accompanying drawings.

The present invention relates to a connecting device 10 for a skin attaching article and, specifically, to a connecting device 10 for a skin attaching article in which the connecting device 10, which is shaped as a clamp, clamps the skin attaching article 20, which may be formed in various shapes and, in the state, a protrusion 14 contacts the skin attaching article 20 and applies a micro current for skin cell exercise.

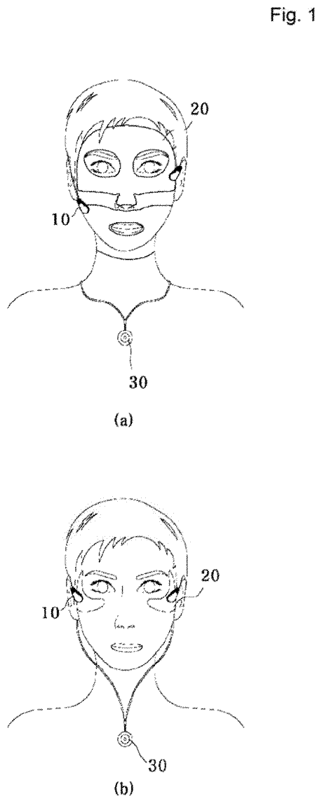

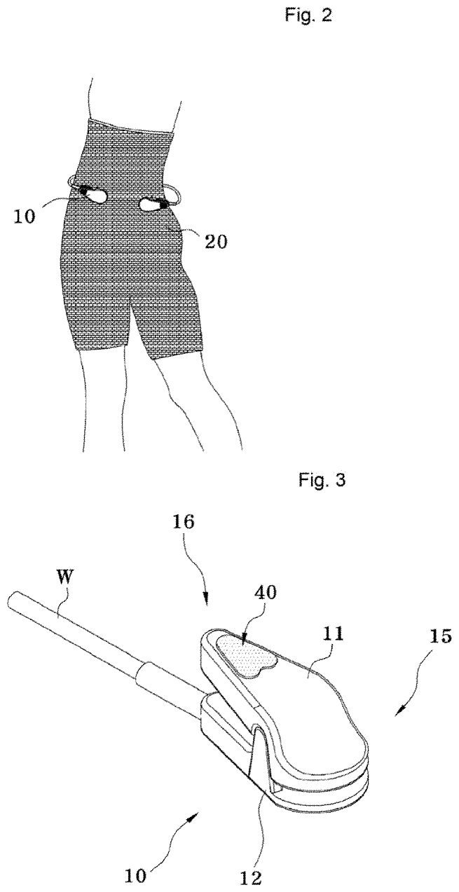

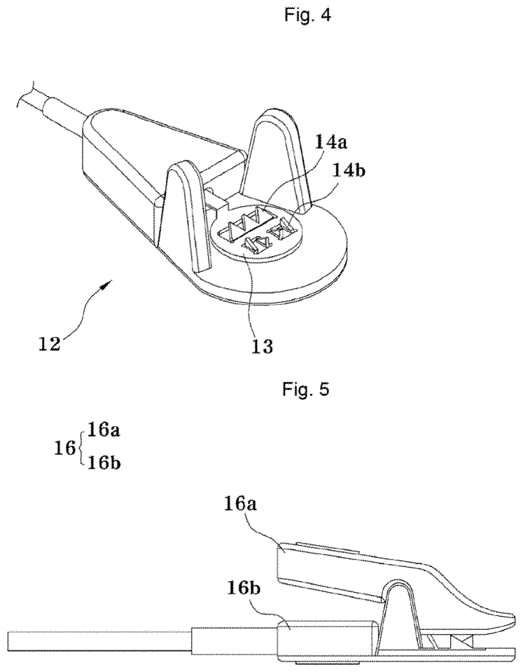

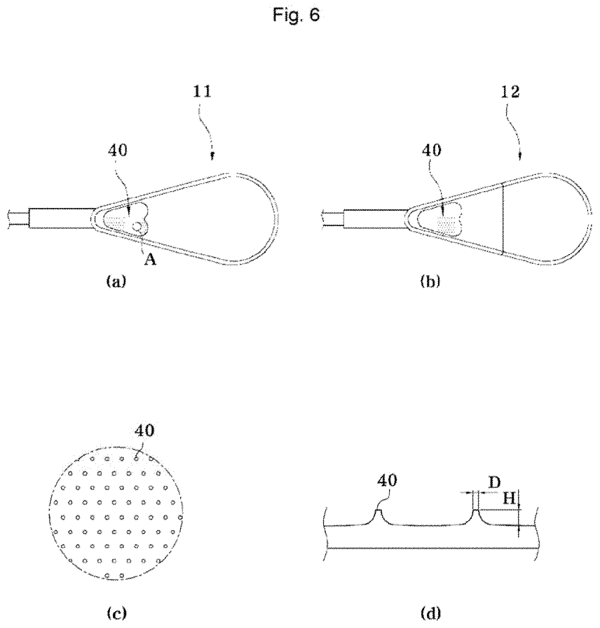

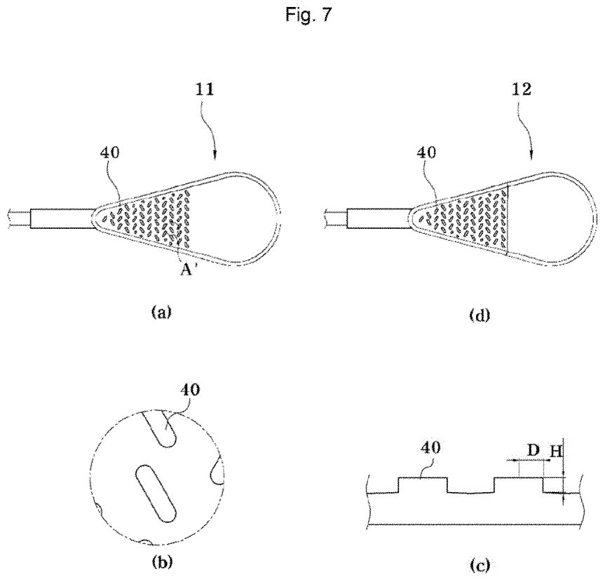

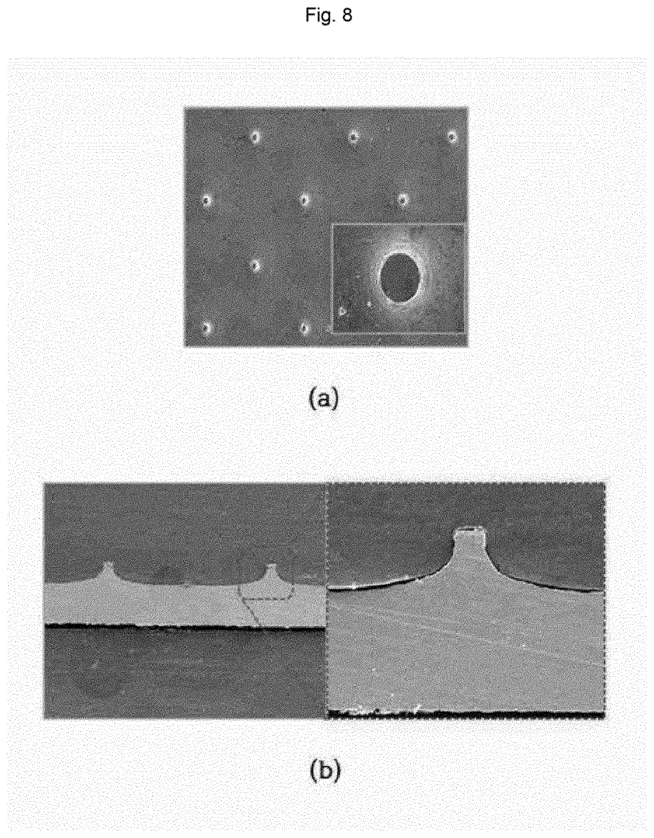

FIG. 1 is a view illustrating an example of wearing a connecting device according to an embodiment of the present invention. FIG. 2 is a view illustrating a skin attaching article according to another embodiment of the present invention. FIG. 3 is a perspective view illustrating a connecting device according to the present invention. FIG. 4 is a partial perspective view illustrating a connecting device according to the present invention. FIG. 5 is a side view illustrating a connecting device according to the present invention. FIG. 6 is a view illustrating anti-slip protrusions according to an embodiment of the present invention. FIG. 7 is a view illustrating anti-slip protrusions according to another embodiment of the present invention, wherein (a) is a plan view, (b) is a rear view, (c) is a partially enlarged view, and (d) is a cross-sectional view. FIG. 8 is a partially enlarged photo of prototype anti-slip protrusions according to the present invention, wherein (a) is a plan view, and (b) is a cross-sectional view.

Referring to FIG. 1, according to the present invention, a connecting device 10 applies power received from a power supply device 30 to a skin attaching article 20. The connecting device 10 is shaped as a clamp. A connecting part 13 is provided in an inside of the connecting device 10. The contact part 13 contacts the skin attaching article 20 and applies the power received from the power supply device 30 to the skin attaching article 20. A handle 16 of the connecting device 10 has anti-slip protrusions 40 to prevent slips.

Specifically, according to the present invention, an upper body 11 and lower body 12, which have a predetermined area, are hinged together on a rotation shaft. A coupling part 15 is provided ahead of the rotation shaft to be coupled to the skin attaching article 20, and a handle 16 is provided behind the rotation shaft to allow the user's finger to contact.

An elastic part S is provided between the upper body 11 and the lower body 12. When no external force is applied to the connecting device 10, the coupling part 15 is in a closed state, i.e., the upper body 11 and the lower body 12 remains contacting each other. If the handle 16a and 16b is pressurized, the coupling part 15 is opened, i.e., the upper body 11 and the lower body 12 are spread from each other.

The elastic part S may be configured of a leaf spring or coil spring to ensure stable operation of the connecting device 10.

The power supply device 30 produces a micro current with a strength similar to the strength of the bio-electricity (which is generated during the human metabolism) and applies the micro current to the skin attaching article 20. According to the present invention, the connecting device 10 receives electric current from the power supply device 30 via an electric wire W, e.g., a lead wire.

Specifically, an end of the electric wire W may be connected with the power supply device 30, and the other end of the electric wire W may be connected with the connecting part 13 through the handle 16b. In other words, a predetermined space may be formed inside the handle 16b. A hole may be formed in the handle 16b to allow the electric wire W to pass through the hole to connect to the connecting part 13.

The skin attaching article 20 is attached to the user's skin, with ingredients effective for skin cell exercise applied thereto. The skin attaching article 20 may be formed in various shapes to be able to receive electric current from the power supply device 30.

For example, the skin attaching article 20 may be formed of a mask sheet cover the user's entire face as shown in FIG. 1(a), and it may be used with a large amount of skin care liquid applied thereto.

Or, the skin attaching article 20 may be formed of a patch that is attached to a portion of the face as shown in FIG. 1(b). In this case, the skin attaching article 20 may include an insulating layer, a conductive layer, and an adhesive layer which are sequentially stacked one over another from top to down, and a skin care gel may be applied to the skin attaching article 20.

The adhesive layer may be formed of a material with electric conductivity and adhesivity. When attached to the skin, the adhesive layer enables the current applied from the connecting part 13 to be delivered more smoothly.

Or, the skin attaching article 20 may be formed to be wearable on the user as shown in FIG. 2.

The skin attaching article 20 may be formed of a material or fiber typically used to manufacture face masks or patches which are attached to the face. The skin attaching article 20 may be formed of various constituent materials in various forms or shapes depending on where the skin attaching article 20 is to be attached.

The connecting device 10 may be coupled, in a random position, to the skin attaching article 20 formed as described. As the connecting device 10 is coupled to the skin attaching article 20, the connecting part 13 of connecting device 10 may simultaneously contact one or two surfaces of the skin attaching article 20 so that electric current generated from the power supply device 30 may be delivered to the skin attaching article 20.

The connecting part 13 may be provided on one or both of the respective facing surfaces of the upper body 11 and the lower body 12 to allow the electric current received from the power supply device 30 to be applied to the skin attaching article 20.

The connecting part 13 may include protrusions 14a and 14b facing the upper body 11 or the lower body 12 so as to achieve easier application of the electric current received from the power supply device 30 and a more secure coupling to the skin attaching article 20.

The protrusions 14a and 14b may have sharp tips to allow the protrusions 14a and 14b to penetrate the skin attaching article 20 to directly contact the internal conductive layer when the present invention is coupled to the skin attaching article 20.

There may be provided a plurality of protrusions 14a and 14b to provide a more secure coupling to the skin attaching article 20. The protrusions 14a and 14b may be formed in a saw-toothed shape and may be used in various arrangements.

For example, as shown in FIGS. 3 and 4, the protrusions 14a may be arranged side-by-side along the width direction of the lower body 12, with the respective first surfaces of the protrusions 14a facing in a forward or backward direction of the lower body 12, and the respective tips of the protrusions 14a may be inclined towards the back end of the lower body 12.

Thus, when the user pulls the connecting device 10 to the back end, with the connecting device 10 attached to the skin attaching article 20, the protrusions 14a may securely bite the skin attaching article 20, preventing separation between the connecting device 10 and the skin attaching article 20.

Or, as shown in FIGS. 3 and 4, the protrusions 14b may be arranged apart from each other along the width direction of the lower body 12, with the respective first surfaces of the protrusions 14b facing in the with direction of the lower body 12.

If the connecting device 10 of the present invention is repeated used for the skin attaching article 20 which is of a patch type, the protrusions 14b may scratches the adhesive layer of the skin attaching article 20 and thus a buildup of adhesive material may arise on the protrusions 14b.

This may significantly deteriorate the conductivity of the protrusions 14b as compared with when it was first used, thus resulting in a lowering of skin care effects. Further, a buildup of dust or other foreign bodies along with the adhesive material would result in an insanitary condition.

Thus, the respective first surfaces of the protrusions 14b may be rendered to face in the left or right direction of the connecting part 13. By so doing, the area where the protrusions 14b rub against the skin attaching article 20 may be reduced, thus reducing the amount of adhesive material chipped out by scratches while preventing a buildup of the adhesive material on one or both surfaces of each saw-toothed protrusion.

The protrusions 14b may include pairs of saw teeth in which the two adjacent saw teeth in each pair are tipped in opposite directions so as to enable a more secure fastening to the skin attaching article 20.

Although FIGS. 4 and 5 illustrate a combination of the above-described different types of protrusions 14a and 14b, this is merely an example, and the present invention is not limited thereto, but other various embodiments may also be possible.

The anti-slip protrusions 40 may be provided to prevent the user's finger from slipping off the connecting device 10. There may be provided a plurality of anti-slip protrusions 40 which protrude from the outside surface of the clamp-shaped handle 16. Specifically, as shown in FIG. 5, the anti-slip protrusions 40 may be formed on each of the top of the upper body 11 and the bottom of the lower body 12 in the handle 16 which are touched by the user's fingers.

In this case, the anti-slip protrusions 40 may be formed in any shape but, to provide more friction with the skin and enhance anti-slip functionality, the tips of the anti-slip protrusions 40 may be formed to be flat.

For example, as shown in FIG. 6 or 7, each anti-slip protrusion 40 may be shaped as a pole which extends upwards, with the top and sides angled therebetween. As shown in FIG. 6, the anti-slip protrusions 40 may be separately manufactured and then attached to the handle 16. Or, as shown in FIG. 7, the anti-slip protrusions 40 may be formed to stick up directly from the handle 16.

Due to the need for providing anti-slip capability even in contact with the skin care liquid-applied skin, the anti-slip protrusions 40 are formed of a material with a predetermined degree of surface roughness.

Meanwhile, the present invention prevents slips by the frictional force that the anti-slip protrusions 40 provide. If less than four anti-slip protrusions 40 are formed per 1 mm², a frictional force may not be stably generated between the skin and the anti-slip protrusion 40. Thus, four or more anti-slip protrusions 40 may be formed per 1 mm².

The height H of each anti-slip protrusion 40 may range from 100 μm to 500 μm.

How deep the anti-slip protrusions 40 intrude into the skin varies depending on the height H of each anti-slip protrusion 40. If the height H is less than 100 μm, the anti-slip effects may be lowered and, if the height H is more than 100 μm, the user may feel pain.

The diameter D of the tip of each anti-slip protrusion 40 may range from 30 μm to 200 μm.

The tip of the anti-slip protrusion 40 comes in surface contact with the user's skin. If the diameter D is less than 30 μm, the area of surface contact reduces, causing it difficult to form a sufficient frictional force. If the diameter D exceeds 200 μm, the distance to the neighbor anti-slip protrusion 40 may be reduced, causing it difficult to create a sufficient frictional force.

By the above configuration, the connecting device 10 may securely be fastened to the skin attaching article 20 simply by allowing the upper body 11 and the lower body 12 to hold the skin attaching article 20. As the protrusion 14 of the connecting part 13 penetrates and couples to the skin attaching article 20, electric current may smoothly be applied to the skin attaching article 20 even without use of a separate coupling means for connection to the skin attaching article 20 unlike the conventional art. Thus, noticeable cost savings are achieved.

Although the user holds the present invention with the slippery skin care liquid on his hands, after connecting with the skin attaching article 20, the anti-slip protrusion 40 prevents the user's fingers from slipping off, enabling the user to press the clamp in a stable position.

Specifically, if the user's hand contacts the anti-slip protrusions 40 and presses it, the skin care liquid partially comes from the user's hand to the spaces between the anti-slip protrusions 40, removing the slippery liquid from the user's hand. Thus, the liquid-freed hand skin comes in surface contact with the tips of the anti-slip protrusions 40, generating a frictional force.

Simultaneously, by the force of pressing the anti-slip protrusions 40, the tips of the anti-slip protrusions 40 are led and stuck to the valleys of the fingerprint of the user's finger, preventing slips.

In other words, the anti-slip protrusions 40 with a simplified structure and the frictional force between the anti-slip protrusions 40 and the finger skin allow the connecting device 10 to be used in a slip-free, stable position.

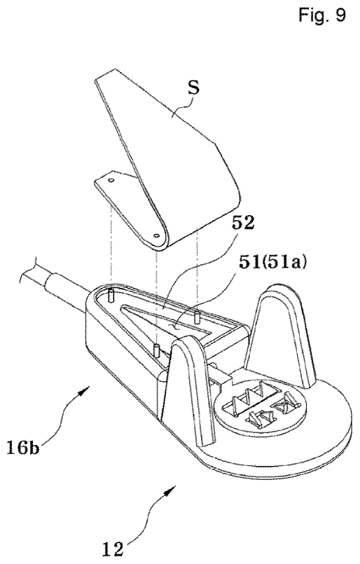

FIGS. 9, 10, 11, and 12 are views illustrating example handles according to various embodiments of the present invention.

The connecting device 10 needs to be configured so that the electric wire W is easily connected with the connecting part 13 and, after connection, they are protected safely.

Thus, the handle 16b facing the upper body 11 has an opening 51 inside, and the opening 51 is selectively opened or closed by a cover 53.

The coupling structure between the opening 51 and the cover 53 may be formed in various manners. In the following description, the terms “first” and “second” or the denotations “a” and “b” are used to clearly distinguish between their respective corresponding components according to embodiments.

For example, as shown in FIG. 9, a first opening 51a is formed in the top of the handle 16b, and a seat 52 internally projecting is formed around the first opening 51a

In this case, the elastic part S is seated on the top of the seat 52 to air-tightly seal the first opening 51a off. Thus, the elastic part S may adjust the operation of the connecting device 10 and protect, or open, the inside of the handle 16b.

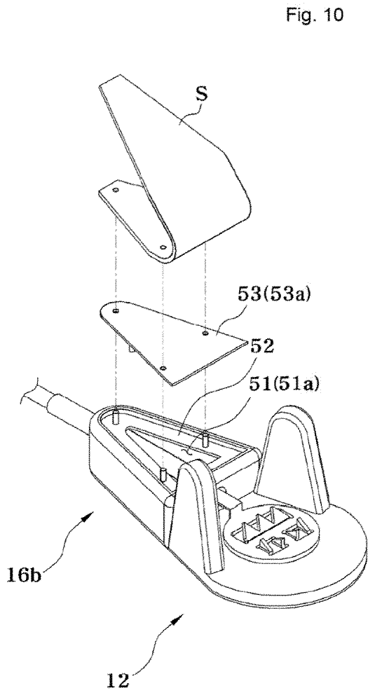

Or, as shown in FIG. 10, a first cover 53a may further be provided on the first opening 51a to selectively open or close the internal space of the handle 16b. In other words, the first cover 53a may air-tightly seal the internal space of the handle 16b off, thereby more stably protecting the internal components.

As operated with the elastic part S seated on the top of the first cover 53a,

the first cover 53a may remain in a secure, fixed position without being separated or escaping off the handle 16b.

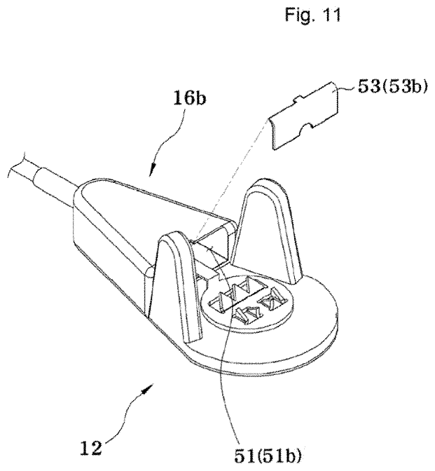

Or, as shown in FIG. 11, the present invention may be formed so that a second cover 53b is separated and coupled from/to an internal side wall of the handle 16b. Specifically, a second opening 51b is formed in the internal side wall of the handle 16b, and the second cover 53b is provided on the second opening 51b to adjust the opening/closing of the internal space of the handle 16b.

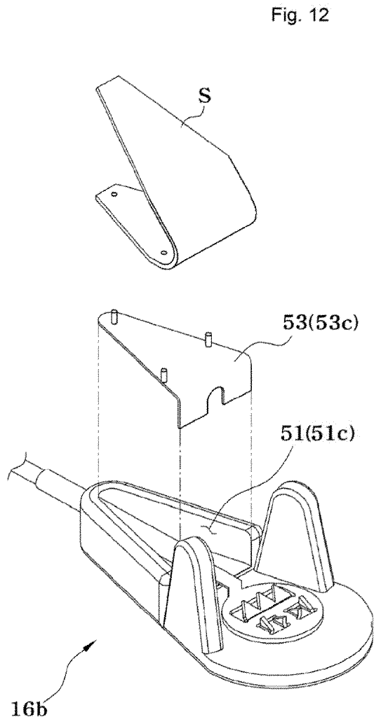

Or, as shown in FIG. 12, a third opening 51c may be formed by connecting the first opening 51a and the second opening 51b, and a cover 53c may be provided on the third opening 51c to adjust the opening/closing of the internal space of the handle 16b.

As set forth above, according to the present invention, as the cover 53 is formed inside the clamp, upon using the connecting device, the cover 53 prevents an external pressure from being directly applied. This may prevent the cover 53 from being unnecessarily opened or damaged and, only when necessary, allows the internal space of the handle 16b to be opened to be used.

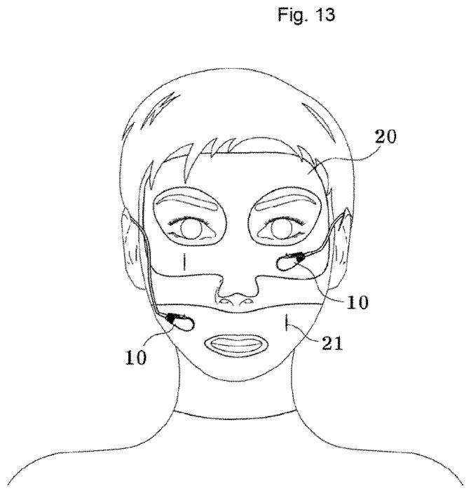

FIG. 13 is a view illustrating an example of wearing a connecting device according to another embodiment of the present invention.

As shown in FIG. 1, upon use, the present invention may be attached to an edge of the skin attaching article 20. However, in this case, the edge where the connecting device 10 is attached in the skin attaching article 20 may fail to tightly contact to the skin but rather float from the skin. Thus, if the user moves, with the skin attaching article 20 attached, the skin attaching article 20 may easily escape off the skin.

Thus, a coupling hole 21 may be formed in the skin attaching article 20 to allow the lower body 12 of the connecting device 10 to pass through, allowing the skin attaching article 20 to remain stable in tight contact with the skin.

Specifically, the coupling hole 21 may be formed of a cut long enough to allow the lower body 12 of the connecting device 10 to be inserted therethrough. The lower body 12 passes through the coupling hole 21 and is inserted, and is connected with the skin attaching article 20.

In other words, the connecting device 10 may be coupled on the surface of the skin attaching article 20, and the skin attaching article 20 may be overall brought in tight contact with the skin. Thus, skin care effects may be further enhanced and, upon use, the skin attaching article 20 may be prevented from escaping off. Thus, more user convenience is achieved.

While the present invention has been shown and described with reference to exemplary embodiments thereof, it will be apparent to those of ordinary skill in the art that various changes in form and detail may be made thereto without departing from the spirit and scope of the present invention as defined by the following claims.

1. A connecting device for a skin attaching article, for applying a micro current for skin cell exercise, the connecting device applying power received from a power supply device (30) to a skin attaching article (20), the connecting device (10) shaped as a clamp, the connecting device having a connecting part (13) in an inside thereof, wherein the contacting part (13) contacts the skin attaching article (20) to apply the power received from the power supply device (30) to the skin attaching article (20).

2. The connecting device of claim 1, wherein a handle (16) of the connecting device (10) has a plurality of anti-slip protrusions (40) projecting from an outside surface.

3. The connecting device of claim 2, wherein each of the anti-slip protrusions (40) has a flat end.

4. The connecting device of claim 1, wherein a coupling hole (21) is formed in the skin attaching article (20) to allow the connecting device (10) to pass therethrough.

5. The connecting device of claim 1, wherein a handle (16b) of the connecting device (10) includes an opening (51) formed in the handle (16b) towards an upper body (11) and a cover (53) configured to couple to, and decouple from, the opening (51) to close or open an internal space of the handle (16b).

6. The connecting device of claim 1, wherein a handle (16b) of the connecting device (10) includes an opening (51) formed in an inside of the handle (16b) towards an upper body (11), and wherein the opening (51) is configured to be opened or closed by an elastic part (S) used for clamping by the connecting device (10).
